(19) United States
(12) Patent Application Publication
(10) Pub. No.: US 2004/0241197 A1
(43) Pub. Date: Dec. 2, 2004

(54) Cosmetic or dermatological preparations including creatinine or a derivative thereof and creatine or a derivative thereof and methods of applying the preparations to the skin

(75) Inventors: Helga Biergiesser, Reinbek (DE); Thomas Blatt, Wedel (DE); Inge Kruse, Hamburg (DE); Holger Lenz, Hamburg (DE); Claudia Mundt, Bremen (DE); Melanie Schmidt, Hamburg (DE); Volker Schreiner, Hamburg (DE); Uwe Schonrock, Nahe (DE); Franz Stab, Echem (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/767,962

(22) Filed: Jan. 26, 2004

Related U.S. Application Data
(63) Continuation of application No. PCT/EP02/08124, filed on Jul. 22, 2002.
(63) Continuation of application No. PCT/EP02/08125, filed on Jul. 22, 2002.

(30) Foreign Application Priority Data
Jul. 25, 2001 (DE) 101 36 077.0

(51) Int. Cl. A61K 7/00

(52) U.S. Cl. 424/401

(57) ABSTRACT

The invention is a cosmetic or dermatological preparation containing a combination of active ingredients consisting of creatinine or a derivative thereof and creatine or a derivative thereof. The cosmetic or dermatological preparation can be applied to the skin for treatment or prophylaxis or various skin conditions or can be used for other purposes.

### CROSS-REFERENCE TO RELATED APPLICATIONS

This application is a continuation of PCT/EP02/08124 filed Jul. 22, 2002 and of PCT/EP02/08125 filed Jul. 22, 2002, both of which are incorporated herein by reference in their entirety. This application also claims the benefit of German Priority Application No. 101 36 077.0, filed Jul. 25, 2001.

### FIELD OF THE INVENTION

The present invention relates to the use of combinations of creatinine and/or creatinine derivatives with creatine and/or its derivatives in cosmetic or dermatological preparations for the treatment and prophylaxis of the symptoms of UV- and/or ozone-induced skin damage, and of inflammatory and degenerative skin conditions.

### BACKGROUND OF THE INVENTION

Cosmetic skin care is primarily understood as meaning that the natural function of the skin as a barrier against environmental influences (e.g. dirt, chemicals, microorganisms) and against the loss of substances intrinsic to the body (e.g. water, natural fats, electrolytes) is strengthened or restored.

Impairment of this function may lead to increased resorption of toxic or allergenic substances or to attack by microorganisms, resulting in toxic or allergic skin reactions.

Another aim of skin care is to compensate for the loss by the skin of sebum and water caused by daily washing. This is particularly important if the natural regeneration ability is inadequate. Furthermore, skincare products should protect against environmental influences, in particular against sun and wind, and delay skin aging.

Chronological skin aging is caused, for example, by endogenous, genetically determined factors. The following structural damage and functional disorders, which can also fall under the term “senile xerosis”, arise, for example, in the epidermis and dermis as the result of aging:

a) Dryness, roughness and formation of dryness wrinkles,

b) Itching and

c) Reduced refatting by sebaceous glands (e.g. after washing).

Exogenous factors, such as UV light and chemical noxae, can have a cumulative effect and, for example, accelerate or supplement the endogenous aging processes. In the epidermis and dermis, for example, the following structural damage and functional disorders may arise in the skin in particular as a result of exogenous factors; these are more far-reaching than the degree and quality of the damage in the case of chronological aging:

d) Visible vascular dilation (teleangiectases, cuperosis);

e) Flaccidity and formation of wrinkles;

f) Local hyperpigmentation, hypopigmentation and abnormal pigmentation (e.g. age spots) and

g) Increased susceptibility to mechanical stress (e.g. cracking).

### SUMMARY OF THE INVENTION

The present invention relates in particular to products for the care of skin aged in a natural way, and for the treatment of the damage caused by photoaging, in particular of the phenomena listed under a) to g).

Products for the care of aged skin are known per se. They comprise, for example, retinoids (vitamin A acid and/or derivatives thereof) or vitamin A and/or derivatives thereof. Their effect on structural damage is, however, limited as regards extent. Furthermore, in product development, there are considerable difficulties in stabilizing the active ingredients to an adequate extent against oxidative decay. The use of products containing vitamin A acid, moreover, often causes severe erythematous skin irritations. Retinoids can therefore only be used in low concentrations.

In particular, the present invention relates to cosmetic preparations having effective protection against harmful oxidation processes in the skin, but also for the protection of cosmetic preparations themselves or for the protection of the constituents of cosmetic preparations against harmful oxidation processes.

The harmful effect of the ultraviolet part of solar radiation on the skin is generally known. Whereas rays with a wavelength of less than 290 nm (the so-called UVC region) are absorbed by the ozone layer in the earth's atmosphere, rays in the range between 290 nm and 320 nm, the so-called UVB region, cause erythema, simple sunburn or even burns of greater or lesser severity.

A maximum erythema activity of sunlight is given as the relatively narrow range around 308 nm.

Numerous compounds are known for protecting against UVB radiation; these are derivatives of 3-benzylidenecamphor, of 4-aminobenzoic acid, of cinnamic acid, of salicylic acid, of benzophenone and also of 2-phenylbenzimidazole.

It is also important to have available filter substances for the range between about 320 nm and about 400 nm, the so-called UVA region, since its rays can cause reactions in cases of photosensitive skin. It has been found that UVA radiation leads to damage of the elastic and collagen fibers of connective tissue, which leads to premature aging of the skin, and is to be regarded as a cause of numerous phototoxic and photoallergic reactions. The harmful effect of UVB radiation can be intensified by UVA radiation.

To protect against rays of the UVA region, therefore, certain derivatives of dibenzoylmethane are used, the photostability of which is inadequate (Int. J. Cosm. Science 10, 53 (1988)).

The UV radiation can, however, also lead to photochemical reactions, in which case the photochemical reaction products again intervene in the skin's metabolism.

Such photochemical reaction products are primarily free-radical compounds, for example hydroxyl radicals. Undefined free-radical photo products, which form in the skin itself, can also display uncontrolled secondary reactions due to their high reactivity. However, singlet oxygen, a non-free-radical excited state of the oxygen molecule, can also arise during UV irradiation, as can short-lived epoxides and many others. Singlet oxygen, for example, differs from triplet oxygen (free-radical ground state), which is normally present, by virtue of its increased reactivity. However, excited, reactive (free-radical) triplet states of the oxygen molecule also exist.

UV radiation is also a type of ionizing radiation. There is therefore the risk that ionic species will also form during UV exposure, which then for their part are able to intervene oxidatively in the biochemical processes.

In order to prevent these reactions, additional antioxidants and/or free-radical scavengers can be incorporated into the cosmetic or dermatological formulations.

It has already been proposed to use vitamin E, a substance with known antioxidative action, in light protection formulations, although, here too, the effect achieved falls a long way short of expectations.

The object of the invention was therefore also to provide cosmetic, dermatological and pharmaceutical active ingredients and preparations, and light protection formulations which serve for the prophylaxis and treatment of photosensitive skin, in particular photodermatoses, preferably PLD.

Other names for polymorphous photodermatosis are PLD, PLE, Mallorca acne and a large number of other names, as given in the literature (e.g. A. Voelckel et al, Zentralblatt Haut- und Geschlechtskrankheiten (1989), 156, p. 2).

Antioxidants are mainly used as substances which protect against the deterioration of the preparations in which they are present. Nevertheless, it is known that in human or animal skin as well, undesired oxidation processes may occur. Such processes play an essential role in skin aging.

The essay “Skin diseases associated with oxidative injury” in “Oxidative stress in dermatology”, p. 323 ff. (Marcel Decker Inc., New York, Basle, Hong Kong, editor: Jürgen Fuchs, Frankfurt, and Lester Packer, Berkeley/California) discusses oxidative skin damage and its more likely causes.

Also for the reason of preventing such reactions, antioxidants and/or free-radical scavengers can be additionally incorporated into cosmetic or dermatological formulations.

Although a number of antioxidants and free-radical scavengers are known, for example, U.S. Pat. Nos. 4,144,325 and 4,248,861, and numerous other documents have already proposed the use of vitamin E, a substance with known antioxidative action in light protection formulations, the effect achieved nevertheless falls a long way short of the desired effect.

It was thus an object of the present invention to find ways to avoid the disadvantages of the prior art. In particular, the effect of remedying the damage associated with endogenous, chronological and exogenous skin aging and the prophylaxis should be permanent, sustained and without the risk of side effects.

To overcome these shortcomings was the object of the present invention.

It has surprisingly been found that the use of creatinine and/or creatinine derivatives with creatine and/or its derivatives in cosmetic or dermatological preparations for the treatment and prophylaxis of the symptoms of UV- and/or ozone-induced skin damage, and of inflammatory and degenerative skin conditions overcomes the disadvantages of the prior art, and at the same time the content of creatinine increases the stability of creatine in cosmetic or dermatological preparations.

### DETAILED DESCRIPTION OF THE PREFERRED EMBODIMENTS

Creatinine (from the Greek: τo κρεαζ=“the meat”) is characterized by the following structure:

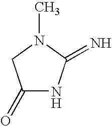

and is produced in the organism as a result of nonenzymatic conversion from creatine phosphate according to

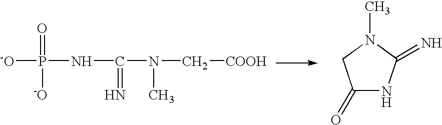

and is excreted via the kidneys. The amount of creatinine excretion is proportional to the muscle mass and is approximately constant for a particular individual. Creatinine is present in meat extract and meat stock cubes.

Creatine (likewise from the Greek: τo κρεαζ=“the meat”) is characterized by the following structure:

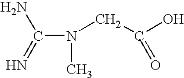

It is found in the myoserum of vertebrates in amounts of 0.05-0.4%, in small amounts also in the brain and blood. In the form of the monohydrate, it is a colorless, crystalline powder. In aqueous solution, creatinine is formed. In the organism, it is formed by the transamidination of L-arginine on glycine to give guanidinoacetic acid and subsequent methylation thereof by means of S-adenosylmethionine (by guanidinoacetate methyltransferase). Creatine is regarded as an appetite-promoting constituent of beef and meat extract. The addition of creatine to the diet enhances physical performance.

Cosmetic or dermatological preparations according to the invention preferably comprise an active ingredient combination of 0.001-50% by weight, particularly preferably 0.01-15% by weight, very particularly preferably 0.1-8% by weight, of creatine and/or creatine derivatives with 0.001-50% by weight, particularly preferably 0.01-15% by weight, very particularly preferably 0.1-8% by weight, of creatinine and/or creatinine derivatives, based on the total composition of the preparations. In this regard, it is advantageous to choose the weight ratio of creatinine to creatine from the range from 50:1 to 1:50, preferably from 10:1 to 1 :10, particularly preferably from 2:1 to 1:2.

A preferred derivative is creatine phosphate, which has the following structure:

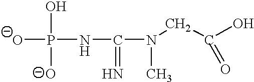

and which is distributed within fresh muscle, where it plays an important role as an energy-storing phosphate (phosphagen). In the working muscle, creatine phosphate gives, with adenosine 5′-diphosphate under the influence of the enzyme creatine kinase, adenosine 5′-triphosphate (ATP) and creatine; in the static muscle, the reverse reaction proceeds.

However, creatine sulfate, creatine acetate, creatine ascorbate and the derivatives esterified on the carboxyl group with mono- or polyfunctional alcohols also lead to advantageous embodiments of the invention.

Although JP2000/247866 describes skin cosmetics with a content of creatine and/or creatinine which can be used in the form of a cream or as a milky lotion, where advantageous skin care properties are attributed to the preparations in question, this specification was unable to pave the way to the present invention.

In addition, WO00/33787 describes the use of creatinine as an effective constituent of deodorants. This specification was unable to pave the way to the present invention either.

Furthermore, EP-A 565 010 describes hair growth and hair coloring preparations with a content of creatinine phosphate. This specification was unable to pave the way to the present invention either.

Finally, U.S. Pat. No. 4,590,067 and EP-A-178 602 describe the use of creatine or creatinine for producing preparations with anti-inflammatory effectiveness. These specifications were unable to pave the way to the present invention either.

The use of the active ingredient combination used according to the invention or cosmetic or topical dermatological preparations with an effective content of active ingredient combination according to the invention surprisingly enables active treatment, but also prophylaxis

of deficient, sensitive or hypoactive skin conditions or deficient, sensitive or hypoactive conditions of skin appendages

of symptoms of premature aging of the skin (e.g. wrinkles, age spots, teleangiecstases) and/or of skin appendages,

of environmentally induced (smoking, smog, reactive oxygen species, free radicals) and, in particular, light-induced negative changes in the skin and the skin appendages

of light-induced skin damage

of pigment disorders,

of itching,

of dry skin conditions and horny layer barrier disorders,

of hair loss and for improved hair growth

of inflammatory skin conditions and also atopic eczema, seborrheic eczema, polymorphous photodermatosis, psoriasis, vitiligo.

The active ingredient combination according to the invention or cosmetic or topical dermatological preparations with an effective content of active ingredient combination according to the invention, however, also surprisingly serves

to calm sensitive or irritated skin

to stimulate the synthesis of collagen, hyaluronic acid and elastin

to stimulate intracellular DNA synthesis, in particular in cases of deficient or hypoactive skin conditions

to increase cell renewal and regeneration of the skin

to increase the skin's own protective and repair mechanisms (for example for dysfunctional enzymes, DNA, lipids, proteins)

for the pre- and post-treatment in cases of topical application of laser and abrasive treatments, which serve, for example, to reduce skin wrinkles and scars, to counteract the resulting skin irritations and to promote the regeneration processes in the damaged skin.

In particular, according to the invention, it is extremely advantageous to use the active ingredient combination used according to the invention or cosmetic or topical dermatological preparations with an effective content of active ingredient combination used according to the invention for the cosmetic or dermatological treatment or prophylaxis of undesired skin conditions.

According to the invention, customary antioxidants can be used in preparations which comprise the active ingredient combinations according to the invention.

The antioxidants are advantageously chosen from the group consisting of amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine-sulfoximines, homocysteine-sulfoximine, buthionine sulfones, penta-, hexa- and heptathionine-sulfoximine) in very low tolerated doses (for example pmol to μmol/kg), and furthermore (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, alaninediacetic acid, flavanoids, polyphenols, catechins, vitamin C and derivatives (for example ascorbyl palmitate, Mg ascorbyl phosphate, ascorbyl acetate), tocopherols and derivatives (for example vitamin E acetate), and coniferyl benzoate of benzoin resin, rutic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

The amount of antioxidants (one or more compounds) in the preparations is preferably from 0.001 to 30% by weight, particularly preferably 0.05-20% by weight, in particular 1-10% by weight, based on the total weight of the preparation.

The prophylaxis or the cosmetic or dermatological treatment with the active ingredient combinations used according to the invention or with the cosmetic or topical dermatological preparations having an effective content of active ingredient combination used according to the invention is carried out in the usual manner, namely by applying the active ingredient used according to the invention or the cosmetic or topical dermatological preparations having an effective content of active ingredient used according to the invention to the affected areas of skin.

The active ingredient combination used according to the invention can advantageously be incorporated into customary cosmetic and dermatological preparations which may be in a variety of forms. They can, for example, be a solution, an emulsion of the water-in-oil (W/O) type or of the oil-in-water (O/W) type, or a multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g. in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active ingredient combination used according to the invention into aqueous systems or surfactant preparations for cleansing the skin and the hair.

The person skilled in the art is of course aware that high-quality cosmetic compositions are mostly inconceivable without customary auxiliaries and additives. Examples thereof include bodying agents, fillers, perfume, dyes, emulsifiers, additional active ingredients such as vitamins or proteins, light protection agents, stabilizers, insect repellents, alcohol, water, salts, and antimicrobially, proteolytically or keratolytically active substances etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g. Cosmetics Directive, Foods and Drugs Act).

In this connection, it is likewise advantageous to add the active ingredient combination used according to the invention as an additive to preparations which already comprise other active ingredients for other purposes.

Accordingly, for the purposes of the present invention, cosmetic or topical dermatological compositions can, depending on their formulation, be used, for example, as skin protection cream, cleansing milk, sunscreen lotion, nourishing cream, day or night cream, etc. In some instances it is possible and advantageous to use the compositions according to the invention as bases for pharmaceutical formulations.

Also favorable in some instances are cosmetic and dermatological preparations which are in the form of a sunscreen. As well as the active ingredient combination used according to the invention, these preferably additionally comprise at least one UVA filter substance and/or at least one UVB filter substance and/or at least one inorganic pigment.

It is, however, also advantageous for the purposes of the present inventions to provide cosmetic and dermatological preparations whose main purpose is not protection against sunlight, but which nevertheless have a content of UV protection substances. Thus, for example, UV-A and/or UV-B filter substances are usually incorporated into day creams.

Preparations according to the invention can advantageously comprise substances which absorb UV radiation in the UVB region, the total amount of filter substances being, for example, 0.1% by weight to 30% by weight, preferably 0.5 to 10% by weight, in particular 1 to 6% by weight, based on the total weight of the preparations.

The UVB filters can be oil-soluble or water-soluble. Examples of oil-soluble substances are:

3-benzylidenecamphor and derivatives thereof, e.g. 3-(4-methylbenzylidene)-camphor,

4-aminobenzoic acid derivatives, preferably 2-ethylhexyl 4-(dimethylamino)-benzoate, amyl 4-(dimethylamino)benzoate;

esters of cinnamic acid, preferably 2-ethylhexyl 4-methoxycinnamate, isopentyl 4-methoxycinnamate;

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;

esters of benzalmalonic acid, preferably di(2-ethylhexyl) 4-methoxybenzal-malonate;

2,4,6-trianilino(p-carbo-2′-ethyl-1′-hexyloxy)-1,3,5-triazine.

Advantageous water-soluble substances are:

2phenylbenzimidazole-5sulfonic acid and salts thereof, e.g. sodium, potassium or triethanolammonium salts;

sulfonic acid derivatives of benzophenones, preferably 2-hydroxy-4-methoxybenzo-phenone-5-sulfonic acid and its salts; sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and its salts.

The list of said UVB filters which can be used according to the invention is of course not intended to be limiting.

The invention also provides the combination of a UVA filter according to the invention with a UVB filter or a cosmetic or dermatological preparation according to the invention which also comprises a UVB filter.

It can also be advantageous to use UVA filters which are customarily present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropyl-phenyl)propane-1,3-dione. Preparations which comprise these combinations are also provided by the invention. It is possible to use the same amounts of UVA filter substances which have been given for UVB filter substances.

Cosmetic and/or dermatological preparations for the purposes of the present invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the amounts given for the above combinations.

The cosmetic and dermatological preparations according to the invention can comprise cosmetic active ingredients, auxiliaries and/or additives as are customarily used in such preparations, e.g. antioxidizing agents, preservatives, bactericides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, solvents which may be used are:

water or aqueous solutions;

oils, such as triglycerides of capric or caprylic acid, but preferably castor oil;

fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;

alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.

The oil phase of the emulsions, oleogels or hydrodispersions or lipodispersions for the purposes of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty acid triglycerides can, for example, be advantageously chosen from the group of synthetic, semisynthetic and natural oils, e.g. olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, and dicaprylyl ether.

Particularly advantageous mixtures are those of C₁₂₋₁₅-alkyl benzoate and 2-ethylhexyl isostearate, those of C₁₂₋₁₅-alkyl benzoate and isotridecyl isononanoate, and those of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or can consist entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexamethylcyclotrisiloxane, polydimethylsiloxane and poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol and, in particular, one or more thickeners which can advantageously be chosen from the group consisting of silicon dioxide, aluminum silicates, polysaccharides and derivatives thereof, e.g. hyaluronic acid, xanthan gum, hydroxypropylmethylcellulose, particularly advantageously from the group of polyacrylates, preferably a polyacrylate from the group of Carbopols, for example Carbopol grades 980, 981, 1382, 2984, 5984, in each case individually or in combination.

Gels used according to the invention usually comprise alcohols of low carbon number, e.g. ethanol, isopropanol, 1,2-propanediol, glycerol and water or an abovementioned oil in the presence of a thickener which, in the case of oily alcoholic gels, is preferably silicon dioxide or an aluminum silicate, and, in the case of aqueous-alcoholic or alcoholic gels, is preferably a polyacrylate.

Solid sticks comprise, for example, natural or synthetic waxes, fatty alcohols or fatty acid esters.

Customary bases which are suitable for use as cosmetic sticks for the purposes of the present invention are liquid oils (e.g. paraffin oils, castor oil, isopropyl myristate), semisolid constituents (e.g. petroleum jelly, lanolin), solid constituents (e.g. beeswax, ceresine and microcrystalline waxes and ozokerite) and high-melting waxes (e.g. carnauba wax, candelilla wax).

Suitable propellants for cosmetic and/or dermatological preparations which can be sprayed from aerosol containers for the purposes of the present invention are the customary known readily volatile, liquefied propellants, for example hydrocarbons (propane, butane, isobutane), which can be used alone or in a mixture with one another. Compressed air can also be used advantageously.

The person skilled in the art is of course aware that there are propellants which are nontoxic per se and are in principle suitable for realizing the present invention in the form of aerosol preparations, but which must nevertheless be avoided because of their unacceptable impact on the environment or other accompanying circumstances, in particular fluorinated hydrocarbons and chlorofluorocarbons (CFCs). For the purposes of the present invention, cosmetic preparations can also be in the form of gels which, in addition to an effective content of the active ingredient according to the invention and solvents customarily used therefor, preferably water, also comprise organic thickeners, e.g. gum arabic, xanthan gum, sodium alginate, cellulose derivatives, preferably methylcellulose, hydroxymethylcellulose, hydroxyethylcellulose, hydroxy-propylcellulose, hydroxypropylmethylcellulose or inorganic thickeners, e.g. aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The examples below serve to illustrate the present invention.

1. PIT emulsions

| | 1 | 2 | 3 | 4 | 5 |
|---|---|---|---|---|---|
| Glycerol monostearate, self- | 0.5 | | 3 | 2 | 4 |
| emulsifying | | | | | |
| Polyoxyethylene(12) cetylstearyl ether | | 5 | | 1 | 1.5 |
| Polyoxyethylene(20) cetylstearyl ether | | | | 2 | |
| Polyoxyethylene(30) cetylstearyl ether | 5 | | 1 | | |
| Stearyl alcohol | | | 3 | | 0.5 |
| Cetyl alcohol | 2.5 | 1 | | 1.5 | |
| 2-Ethylhexyl methoxycinnamate | | | | 5 | 8 |
| 2,4-Bis(4-(2-ethylhexyloxy)-2- | | 1.5 | | 2 | 2.5 |
| hydroxyl)phenyl)-6- | | | | | |
| (4-methoxyphenyl)-(1,3,5)-triazine | | | | | |
| 1-(4-tert-Butylphenyl)-3- | | | 2 | | |
| (4-methoxyphenyl)- | | | | | |
| 1,3-propanedione | | | | | |
| Diethylhexylbutamidotriazone | 1 | 2 | | 2 | |
| Ethylhexyltriazone | 4 | | 3 | 4 | |
| 4-Methylbenzylidenecamphor | | 4 | | | 2 |
| Octocrylene | | 4 | | | 2.5 |
| Phenylene-1,4-bis(monosodium, 2- | | | 0.5 | | 1.5 |
| benzimidazyl-5,7-disulfonic acid | | | | | |
| Phenylbenzimidazolesulfonic acid | 0.5 | | | 3 | |
| C12-15 alkyl benzoate | | 2.5 | | | 5 |
| Titanium dioxide | 0.5 | 1 | | 3 | 2 |
| Zinc oxide | 2 | | 3 | 0.5 | 1 |
| Dicaprylyl ether | | | 3.5 | | |
| Butylene glycol dicaprylate/dicaprate | 5 | | | 6 | |
| Dicaprylyl carbonate | | | 6 | | 2 |
| Dimethicone polydimethylsiloxane | | 0.5 | 1 | | |
| Phenylmethylpolysiloxane | 2 | | | 0.5 | 0.5 |
| Shea butter | | 2 | | | 0.5 |
| PVP hexadecene copolymer | 0.5 | | | 0.5 | 1 |
| Glycerol | 3 | 7.5 | 5 | 7.5 | 2.5 |
| Tocopherol acetate | 0.5 | | 0.25 | | 1 |
| Creatine | 0.2 | 0.5 | 0.5 | 0.6 | 0.1 |
| Creatinine | 0.1 | 0.5 | 1 | 0.2 | 0.1 |
| Alpha-glucosylrutin | 0.1 | | 0.2 | | |
| Preservative | q.s. | q.s. | q.s. | q.s. | q.s. |
| Ethanol | 3 | 2 | 1.5 | | 1 |
| Perfume | q.s. | q.s. | q.s. | q.s. | q.s. |
| Water | ad | ad | ad | ad | ad |
| | 100 | 100 | 100 | 100 | 100 |

2. Examples of O/W cream

| | Examples | | | | |
|---|---|---|---|---|---|
| | 1 | 2 | 3 | 4 | 5 |
| Glyceryl stearate citrate | | | 2 | | 2 |
| Glyceryl stearate self-emulsifying | 4 | 3 | | | |
| PEG-40 stearate | 1 | | | | |
| Polyglyceryl-3 methylglucose distearate | | | | 3 | |
| Sorbitan stearate | | | | | 2 |
| Stearic acid | | 1 | | | |
| Polyoxyethylene(20) cetylstearyl ether | | | | | |
| Stearyl alcohol | | | 5 | | |
| Cetyl alcohol | 3 | 2 | | 3 | |
| Cetylstearyl alcohol | | | | | 2 |
| C12-15 alkyl benzoate | | | | | |
| Caprylic/capric triglyceride | 5 | 3 | 4 | 3 | 3 |
| Octyldodecanol | | | 2 | | 2 |
| Dicaprylyl ether | | 4 | | 2 | 1 |
| Paraffinum liquidum | 5 | 2 | | 3 | |
| Titanium dioxide | | | 1 | | |
| 4-Methylbenzylidenecamphor | | | 1 | | |
| 1-(4-tert-Butylphenyl)-3-(4-methoxyphenyl)- | | | 0.5 | | |
| 1,3-propanedione | | | | | |
| Creatine | 0.1 | 0.5 | 0.5 | 2 | 0.1 |
| Creatinine | 0.2 | 0.5 | 0.1 | 1 | 0.3 |
| Tocopherol | 0.1 | | | | 0.2 |
| Biotin | | | 0.05 | | |
| Ethylenediaminetetraacetic acid trisodium | 0.1 | | 0.1 | 0.1 | |
| Preservative | q.s. | q.s. | q.s. | q.s. | q.s. |
| Xanthan gum | | | | | |
| Polyacrylic acid | 3 | 0.1 | | 0.1 | 0.1 |
| Sodium hydroxide solution 45% | q.s. | q.s. | q.s. | q.s. | q.s. |
| Glycerol | 5 | 3 | 4 | 3 | 3 |
| Butylene glycol | | 3 | | | |
| Perfume | q.s. | q.s. | q.s. | q.s. | q.s. |
| Water | ad | ad | ad | ad | ad |
| | 100 | 100 | 100 | 100 | 100 |

3. Examples of O/W cream

| | Examples | | | | |
|---|---|---|---|---|---|
| | 6 | 7 | 8 | 9 | 10 |
| Glyceryl stearate citrate | | 2 | 2 | | |
| Glyceryl stearate self-emulsifying | 5 | | | | |
| Stearic acid | | | | 2.5 | 3.5 |
| Stearyl alcohol | 2 | | | | |
| Cetyl alcohol | | | | 3 | 4.5 |
| Cetylstearyl alcohol | | 3 | 1 | | 0.5 |
| C12-15 alkyl benzoate | | 2 | 3 | | |
| Caprylic/capric triglyceride | 2 | | | | |
| Octyldodecanol | 2 | 2 | | 4 | 6 |
| Dicaprylyl ether | | | | | |
| Paraffinum liquidum | | 4 | 2 | | |
| Cyclic dimethylpolysiloxane | | | | 0.5 | 2 |
| Dimethicone polydimethylsiloxane | 2 | | | | |
| Titanium dioxide | 2 | | | | |
| 4-Methylbenzylidenecamphor | 1 | | | | 1 |
| 1-(4-tert-Butylphenyl)-3-(4-methoxyphenyl)- | 0.5 | | | | 0.5 |
| 1,3-propanedione | | | | | |
| Creatine | 0.2 | 0.3 | 1 | 0.5 | 0.8 |
| Creatinine | 0.2 | 0.7 | 0.25 | 1 | 0.4 |
| Tocopherol | | | | | 0.05 |
| Ethylenediaminetetraacetic acid trisodium | | | 0.2 | | 0.2 |
| Preservative | q.s. | q.s. | q.s. | q.s. | q.s. |
| Xanthan gum | | | 0.2 | | |
| Polyacrylic acid | 0.15 | 0.1 | | 0.05 | 0.05 |
| Sodium hydroxide solution 45% | q.s. | q.s. | q.s. | q.s. | q.s. |
| Glycerol | 3 | | 3 | 5 | 3 |
| Butylene glycol | | 3 | | | |
| Ethanol | | 3 | | 3 | |
| Perfume | q.s. | q.s. | q.s. | q.s. | q.s. |
| Water | ad | ad | ad | ad | ad |
| | 100 | 100 | 100 | 100 | 100 |

4. Examples of W/O emulsions

| | 1 | 2 | 3 | 4 | 5 |
|---|---|---|---|---|---|
| Cetyldimethicone copolyol | | 2.5 | | 4 | |
| Polyglyceryl-2 dipolyhydroxystearate | 5 | | | | 4.5 |
| PEG-30 dipolyhydroxystearate | | | 5 | | |
| 2-Ethylhexyl methoxycinnamate | | 8 | | 5 | 4 |
| 2,4-Bis(4-(2-ethylhexyloxy)-2- | 2 | 2.5 | | 2 | 2.5 |
| hydroxyl)phenyl)-6-(4-methoxyphenyl)- | | | | | |
| (1,3,5)-triazine | | | | | |
| 1-(4-tert-Butylphenyl)-3-(4-methoxyphenyl)- | | | 2 | 1 | |
| 1,3-propanedione | | | | | |
| Diethylhexylbutamidotriazone | 3 | 1 | | | 3 |
| Ethylhexyltriazone | | | 3 | 4 | |
| 4-Methylbenzylidenecamphor | | 2 | | 4 | 2 |
| Octocrylene | 7 | 2.5 | 4 | | 2.5 |
| Diethylhexylbutamidotriazone | 1 | | | 2 | |
| Phenylene-1,4-bis(monosodium, 2- | 1 | 2 | 0.5 | | |
| benzimidazyl-5,7-disulfonic acid | | | | | |
| Phenylbenzimidazolesulfonic acid | 0.5 | | | 3 | 2 |
| Titanium dioxide | | 2 | 1.5 | | 3 |
| Zinc oxide | 3 | 1 | 2 | 0.5 | |
| Paraffinum liquidum | | | 10 | | 8 |
| C12-15 alkyl benzoate | | | | 9 | |
| Dicaprylyl ether | 10 | | | | 7 |
| Butylene glycol dicaprylate/dicaprate | | | 2 | 8 | 4 |
| Dicaprylyl carbonate | 5 | | 6 | | |
| Dimethicone polydimethylsiloxane | | 4 | 1 | 5 | |
| Phenylmethylpolysiloxane | 2 | 25 | | | 2 |
| Shea butter | | | 3 | | |
| PVP hexadecene copolymer | 0.5 | | | 0.5 | 1 |
| Octoxyglycerol | | 0.3 | 1 | | 0.5 |
| Glycerol | 3 | 7.5 | | 7.5 | 2.5 |
| Glycine soya | | 1 | 1.5 | | |
| Magnesium sulfate | 1 | 0.5 | | 0.5 | |
| Magnesium chloride | | | 1 | | 0.7 |
| Tocopherol acetate | 0.5 | | 0.25 | | 1 |
| Creatine | 0.4 | 0.1 | 0.5 | 2 | 2 |
| Creatinine | 0.1 | 0.6 | 1 | 1 | 0.8 |
| Preservative | q.s. | q.s. | q.s. | q.s. | q.s. |
| Ethanol | 3 | | 1.5 | | 1 |
| Perfume | q.s. | q.s. | q.s. | q.s. | q.s. |
| Water | ad | ad | ad | ad | ad |
| | 100 | 100 | 100 | 100 | 100 |

5. Examples of W/O emulsions

| | 6 | 7 |
|---|---|---|
| Polyglyceryl-2 dipolyhydroxystearate | 4 | 5 |
| PEG-30 dipolyhydroxystearate | | |
| Lanolin alcohol | 0.5 | 1.5 |
| Isohexadecane | 1 | 2 |
| Myristyl myristate | 0.5 | 1.5 |
| Petroleum jelly | 1 | 2 |
| 1-(4-tert-Butylphenyl)-3-(4-methoxyphenyl)- | 0.5 | 1.5 |
| 1,3-propanedione | | |
| 4-Methylbenzylidenecamphor | 1 | 3 |
| Butylene glycol dicaprylate/dicaprate | 4 | 5 |
| Shea butter | | 0.5 |
| Butylene glycol | | 6 |
| Octoxyglycerol | | 3 |
| Glycerol | 5 | |
| Tocopherol acetate | 0.5 | 1 |
| Creatine | 0.2 | 0.5 |
| Creatinine | 0.2 | 0.25 |
| Trisodium EDTA | 0.2 | 0.2 |
| Preservative | q.s. | q.s. |
| Ethanol | | 3 |
| Perfume | q.s. | q.s. |
| Water | ad 100 | ad 100 |

6. Examples of hydrodispersions

| | 1 | 2 | 3 | 4 | 5 |
|---|---|---|---|---|---|
| Polyoxyethylene(20) cetylstearyl ether | 1 | | | 0.5 | |
| Cetyl alcohol | | | 1 | | |
| Sodium polyacrylate | | 0.2 | | 0.3 | |
| Acrylate/C10-30-alkyl acrylate crosspolymer | 0.5 | | 0.4 | 0.1 | 0.1 |
| Xanthan gum | | 0.3 | 0.15 | | 0.5 |
| 2-Ethylhexyl methoxycinnamate | | | | 5 | 8 |
| 2,4-Bis(4-(2-ethylhexyloxy)-2- | | 1.5 | | 2 | 2.5 |
| hydroxyl)phenyl)-6-(4-methoxyphenyl)- | | | | | |
| (1,3,5)-triazine | | | | | |
| 1-(4-tert-Butylphenyl)-3-(4-methoxyphenyl)- | 1 | | 2 | | |
| 1,3-propanedione | | | | | |
| Diethylhexylbutamidotriazone | | 2 | | 2 | 1 |
| Ethylhexyltriazone | 4 | | 3 | 4 | |
| 4-Methylbenzylidenecamphor | 4 | 4 | | | 2 |
| Octocrylene | | 4 | 4 | | 2.5 |
| Phenylene-1,4-bis(monosodium, 2- | 1 | | 0.5 | | 2 |
| benzimidazyl-5,7-disulfonic acid | | | | | |
| Phenylbenzimidazolesulfonic acid | 0.5 | | | 3 | |
| Titanium dioxide | 0.5 | | 2 | 3 | 1 |
| Zinc oxide | 0.5 | 1 | 3 | | 2 |
| C12-15 alkyl benzoate | 2 | 2.5 | | | |
| Dicaprylyl ether | | 4 | | | |
| Butylene glycol dicaprylate/dicaprate | 4 | | 2 | 6 | |
| Dicaprylyl carbonate | | 2 | 6 | | |
| Dimethicone polydimethylsiloxane | | 0.5 | 1 | | |
| Phenylmethylpolysiloxane | 2 | | | 0.5 | 2 |
| Shea butter | | 2 | | | |
| PVP hexadecene copolymer | 0.5 | | | 0.5 | 1 |
| Octoxyglycerol | | | 1 | | 0.5 |
| Glycerol | 3 | 7.5 | | 7.5 | 2.5 |
| Glycine soya | | | 1.5 | | |
| Tocopherol acetate | 0.5 | | 0.25 | | 1 |
| Creatine | 0.3 | 0.3 | 3 | 1 | 0.2 |
| Creatinine | 0.15 | 0.6 | 1 | 1 | 0.8 |
| Preservative | q.s. | q.s. | q.s. | q.s. | q.s. |
| Ethanol | 3 | 2 | 1.5 | | 1 |
| Perfume | q.s. | q.s. | q.s. | q.s. | q.s. |
| Water | ad | ad | ad | ad | ad |
| | 100 | 100 | 100 | 100 | 100 |

7. Example (gel cream)

| | |
|---|---|
| Acrylate/C10-30 alkyl acrylate crosspolymer | 0.4 |
| Polyacrylic acid | 0.2 |
| Xanthan gum | 0.1 |
| Cetearyl alcohol | 3 |
| C12-15 alkyl benzoate | 4 |
| Caprylic/capric triglyceride | 3 |
| Cyclic dimethylpolysiloxane | 5 |
| Dimethicone polydimethylsiloxane | 1 |
| Creatine | 0.4 |
| Creatinine | 0.2 |
| Glycerol | 3 |
| Sodium hydroxide | q.s. |
| Preservative | q.s. |
| Perfume | q.s. |
| Water | ad 100 |
| pH adjusted to 6.0 | |

8. Example (W/O cream)

| | |
|---|---|
| Polyglyceryl-3 diisostearate | 3.5 |
| Glycerol | 3 |
| Polyglyceryl-2 dipolyhydroxystearate | 3.5 |
| Creatine | 1 |
| Creatinine | 0.5 |
| Preservative | q.s. |
| Perfume | q.s. |
| Water | ad 100 |
| Magnesium sulfate | 0.6 |
| Isopropyl stearate | 2 |
| Caprylyl ether | 8 |
| Cetearyl isononanoate | 6 |

9. Example (W/O/W cream)

| | |
|---|---|
| Glyceryl stearate | 3 |
| PEG-100 stearate | 0.75 |
| Behenyl alcohol | 2 |
| Caprylic/capric triglyceride | 8 |
| Octyldodecanol | 5 |
| C₁₂₋₁₅ alkyl benzoate | 3 |
| Creatine | 2 |
| Creatinine | 1 |
| Magnesium sulfate (MgSO₄) | 0.8 |
| Ethylenediaminetetraacetic acid | 0.1 |
| Preservative | q.s. |
| Perfume | q.s. |
| Water | ad 100 |
| pH adjusted to 6.0 | |

That which is claimed:

1. A cosmetic or dermatological preparation with a content of an active ingredient combination of at least one compound selected from the group consisting of creatinine and derivatives thereof and at least one compound selected from the group consisting of creatine and derivatives thereof.

2. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the prophylaxis or treatment of an inflammatory skin condition or for the protection of sensitive or dry skin.

3. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the prophylaxis or treatment of the symptoms of intrinsic or extrinsic skin aging or of the harmful effects of ultraviolet radiation on the skin.

4. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the treatment or prophylaxis of environmentally induced negative alterations of the skin or skin appendages.

5. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the treatment or prophylaxis of pigmentation disorders.

6. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the treatment or prophylaxis of functional disorders of skin appendages.

7. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the enhancement of hair growth.

8. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for increasing ceramide biosynthesis.

9. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for strengthening the barrier function of the skin.

10. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the treatment or prophylaxis of disorders of the normal skin pH and of the osmolyte balance.

11. The cosmetic or dermatological preparation as claimed in claim 1, wherein the at least one compound selected from the group consisting of creatinine and derivatives thereof and the at least one compound selected from the group consisting of creatine and derivatives thereof are present in an amount effective for the treatment or prophylaxis of deviations from the normal cell-cell communication in the skin.

12. The cosmetic or dermatological preparation as claimed in claim 1, wherein the at least one compound selected from the group consisting of creatinine and derivatives thereof is present in an amount from 0.1 to 10 wt %, based on the total weight of the preparation.

13. The cosmetic or dermatological preparation as claimed in claim 12, wherein the at least one compound selected from the group consisting of creatine and derivatives thereof is present in an amount from 0.1 to 10 wt %, based on the total weight of the preparation.

14. The cosmetic or dermatological preparation as claimed in claim 1, wherein the at least one compound selected from the group consisting of creatine and derivatives thereof is present in an amount from 0.1 to 10 wt %, based on the total weight of the preparation.

15. The cosmetic or dermatological preparation as claimed in claim 1, wherein the at least one compound selected from the group consisting of creatinine and derivatives thereof is present in an amount from 0.1 to 1 wt % and the at least one compound selected from the group consisting of creatine and derivatives thereof is present in an amount from 0.1 to 3 wt %, based on the total weight of the preparation.

16. The cosmetic or dermatological preparation as claimed in claim 1, wherein the weight ratio of the at least one compound selected from the group consisting of creatinine and derivatives thereof to the at least one compound selected from the group consisting of creatine and derivatives thereof is from 1:2 to 2:1.

17. The cosmetic or dermatological preparation as claimed in claim 1, wherein the at least one compound selected from the group consisting of creatine and derivatives thereof comprises at least one compound selected from the group consisting of creatine phosphate, creatine sulfate, creatine acetate, creatine ascorbate, and esters of creatine and mono- or polyfunctional alcohols.

18. The cosmetic or dermatological preparation as claimed in claim 1, wherein the at least one compound selected from the group consisting of creatine and derivatives thereof comprises creatine phosphate.

19. A method for the prophylaxis or treatment of an inflammatory skin condition or for the protection of dry or sensitive skin, comprising applying to the skin a cosmetic or dermatological preparation comprising at least one compound selected from the group consisting of creatinine and derivatives thereof and the at least one compound selected from the group consisting of creatine and derivatives thereof in an amount effective for the prophylaxis or treatment of the inflammatory skin condition or for the protection of sensitive or dry skin.

20. The method as claimed in claim 19, wherein the inflammatory, sensitive or dry skin condition is one or more conditions selected from the group consisting of atopic eczema, seborrheic eczema, polymorphous photodermatosis, psoriasis, vitiligo, wound healing disorders, itching; sensitive or irritated skin, light-induced skin damage and UV-induced immunosuppression, changes in desquamation, changes in the normal fibroblast and keratinocyte proliferation, changes in the normal fibroblast and keratinocyte differentiation of deficient sensitive or hypoactive skin conditions or deficient sensitive or hypoactive conditions of skin appendages, and increased skin thickness.

21. A method for treating the prophylaxis or treatment of the symptoms of intrinsic or extrinsic skin aging or of the harmful effects of ultraviolet radiation on the skin, comprising applying to the skin a cosmetic or dermatological preparation comprising at least one compound selected from the group consisting of creatinine and derivatives thereof and the at least one compound selected from the group consisting of creatine and derivatives thereof in an amount effective for the prophylaxis or treatment of the symptoms of intrinsic or extrinsic skin aging or of the harmful effects of ultraviolet radiation on the skin.

22. The method as claimed in claim 21, wherein the symptoms of intrinsic or extrinsic skin aging and the harmful effects of ultraviolet radiation on the skin are one or more symptoms or harmful effects selected from the group consisting of age spots, wrinkles, teleangiectases, skin sagging, loss of elasticity, disappearance of the epidermal and dermal cell layers, disappearance of the constituents of connective tissue, disappearance of the retinal cones and capillary vessels, skin luster and fatigue, increased activation of proteolytic enzymes in the skin, metalloproteinases, disorders of the normal collagen, hyaluronic acid, elastin and glycosaminoglycan homeostasis, disorders of normal skin regeneration, changes in the normal fibroblast and keratinocyte proliferation, changes in the normal fibroblast and keratinocyte differentiation, and deficiency symptoms of intracellular DNA synthesis.

23. A method for the treatment or prophylaxis of environmentally induced negative alterations of the skin or skin appendages, comprising applying to the skin a cosmetic or dermatological preparation comprising at least one compound selected from the group consisting of creatinine and derivatives thereof and the at least one compound selected from the group consisting of creatine and derivatives thereof in an amount effective for the treatment or prophylaxis of environmentally induced negative alterations of the skin or skin appendages.

24. The method as claimed in claim 23, wherein the environmentally induced negative alterations of the skin or skin appendages are caused by one or more selected from the group consisting of smoking, smog, reactive oxygen species and free radicals.

25. A method for the treatment or prophylaxis of pigmentation disorders, comprising applying to the skin a cosmetic or dermatological preparation comprising at least one compound selected from the group consisting of creatinine and derivatives thereof and the at least one compound selected from the group consisting of creatine and derivatives thereof in an amount effective for the treatment or prophylaxis of pigmentation disorders.

26. A method for the treatment or prophylaxis of functional disorders of skin appendages, comprising applying to the skin a cosmetic or dermatological preparation comprising at least one compound selected from the group consisting of creatinine and derivatives thereof and the at least one compound selected from the group consisting of creatine and derivatives thereof in an amount effective for the treatment or prophylaxis of functional disorders of skin appendages.

27. The method as claimed in claim 26, wherein the functional disorders of skin appendages include one or more selected from the group consisting of hair loss, seborrheic phenomena, greasy skin, greasy hair, comedones, and dandruff.

28. A method for the enhancement of hair growth, comprising applying to the skin a cosmetic or dermatological preparation comprising at least one compound selected from the group consisting of creatinine and derivatives thereof and the at least one compound selected from the group consisting of creatine and derivatives thereof in an amount effective for the enhancement of hair growth.

29. A method for increasing ceramide biosynthesis, comprising applying to the skin a cosmetic or dermatological preparation comprising at least one compound selected from the group consisting of creatinine and derivatives thereof and the at least one compound selected from the group consisting of creatine and derivatives thereof in an amount effective for increasing ceramide biosynthesis.

30. A method for strengthening the barrier function of the skin, comprising applying to the skin a cosmetic or dermatological preparation comprising at least one compound selected from the group consisting of creatinine and derivatives thereof and the at least one compound selected from the group consisting of creatine and derivatives thereof in an amount effective for strengthening the barrier function of the skin.

31. The method as claimed in claim 30, wherein the strengthening of the barrier function of the skin provides prophylaxis or treatment of one or more conditions selected from the group consisting of horny layer barrier disorders, changes in normal lipid peroxidations, changes in transepidermal water loss and changes in the normal moisture content of the skin.

32. A method for the treatment or prophylaxis of disorders of the normal skin pH and of the osmolyte balance, comprising applying to the skin a cosmetic or dermatological preparation comprising at least one compound selected from the group consisting of creatinine and derivatives thereof and the at least one compound selected from the group consisting of creatine and derivatives thereof in an amount effective for the treatment or prophylaxis of disorders of the normal skin pH and of the osmolyte balance.

33. A method for the treatment or prophylaxis of deviations from the normal cell-cell communication in the skin, comprising applying to the skin a cosmetic or dermatological preparation comprising at least one compound selected from the group consisting of creatinine and derivatives thereof and the at least one compound selected from the group consisting of creatine and derivatives thereof in an amount effective for the treatment or prophylaxis of deviations from the normal cell-cell communication in the skin.
